SEQUENCE LISTING (2 sequences)

SEQ ID NO: 1 - DNA, 21 bp, Artificial
  misc_feature: primer for PCR amplificaction
ccagtcccactatctagaaaa

SEQ ID NO: 2 - DNA, 20 bp, Artificial
  misc_feature: primers for PCR amplificaction
atggctcatggctgtgatag

(19) United States
(12) Patent Application Publication
(10) Pub. No.: US 2007/0105095 A1
(43) Pub. Date: May 10, 2007

(54) Process for detecting predisposition to a cardiovascular disease

(76) Inventors: Jordi Fontcuberta, Barcelona (ES); Jose Manuel Soria Fernandez, Barcelona (ES)

Correspondence Address: FAY KAPLUN & MARCIN, LLP, 15O BROADWAY, SUITE 702, NEW YORK, NY, 10038, US

(21) Appl. No.: 10/503,156

(22) PCT Filed: Jan. 30, 2003
(86) PCT No.: PCT/IB03/00315

§ 371 Date: Jul. 28, 2004

(30) Foreign Application Priority Data
Jan. 31, 2002 (ES) P 200200308

(51) Int. Cl. C12Q 1/68

(52) U.S. Cl. 435/6

(57) ABSTRACT

Includes the identification in the genetic material of an isolated biological sample from said human an allelic variant which consist on the substitution of a cytosine by an tymine in the position 46 over the transcriptional origin of Factor XII (46C/T) within the locus of chromosome 5, limited by the D5S400 and D5S408 markers, being the presence of said allelic variant is indicative of a predisposition to a cardiovascular disease. In said process, use is made of a biological sample susceptible of including at least one allelic variant within the chromosome 5 locus limited by the D5S400 and D5S408 markers in order to determine a predisposition to cardiovascular diseases which manifest with thrombotic events. The process permits detection of a predisposition to a cardiovascular disease in a human, so that a suitable preventive an therapeutic strategy can be designed.

### FIELD OF THE INVENTION

The present invention relates to a new process for detecting predisposition to a cardiovascular disease in humans.

### BACKGROUND OF THE INVENTION

Cardiovascular diseases, and in particular both arterial and venous thrombosis, are one of the most frequent causes of mortality in the industrialised countries.

Both genetic and environmental factors are involved in the causes of thrombosis. The high prevalence of thrombosis and the known environmental influence (for example, the use of oral contraceptives) suggest the involvement of many genes in the susceptibility to this disease.

Indeed, several genetic defects leading to an increase in the thrombotic risk have been located and characterized (Lane D A, Mannucci P M, Bauer K A, Bertina R M, Bochkov N P, Boulyjenkov V, Chandy M, Dahlback B, Ginter E K, Miletich J P, Rosendaal F R, Seligsohn U. Inherited Thrombophilia: Part 1. Thromb Haemost 1996; 76: 651-662).

In general, however, very little information is available about the relative importance of the genetic factors in the thrombosis risk of the population. Furthermore, it is unlikely that these known mutations, with their relatively low frequencies, constitute the main cause of thrombosis risk.

Recently, the inventors of the present invention quantified the genetic component of susceptibility to thrombosis and the related phenotypes (Souto J C, Almasy L, Borrell M, Gari M, Marinez E, Mateo J, Stone W H, Blangero J, Fontcuberta J. Genetic determinants of hemostasis phenotypes in Spanish families, Circulation, 101: 1546-1551.2000; Souto J C, Almasy L, Borrell M, Blanco-Vaca F, Mateo J, Soria J M, Coll I, Felices R, Stone W, Fontcuberta J, Blangero J. Genetic susceptibility to thrombosis and its relationship to physiological risk factors: The GAIT study. Am J Hum Genet 67: 1452-1459.2000), it being observed that levels of factor XII show one of the highest heridabilities (67%) and a significant positive genetic correlation (0.351), which indicates that some of the genes which influence the variation of this physiological risk factor also influence the risk of thrombosis.

On the other hand, early diagnosis of this type of diseases is of great interest, especially in those people who, though they have not developed the illness, belong to a risk group due to presenting some genetic alteration, as many of the secondary complications associated with these diseases could be avoided thereby.

The existing lack of knowledge of the genetic causes which influence thrombosis gives rise to problems, however, when it comes to making suitable diagnosis for identification of individuals with a genetic risk of developing cardiovascular diseases, and in particular thrombosis, because, as stated above, this is a multigenic illness (one in which several genes are involved).

The aim of the present invention is to solve the problems in the diagnosis of cardiovascular disease by providing a process which permits identification of at least one allelic variant on a locus of chromosome 5 limited by the D5S400 and D5S408 markers for the identification of individuals who present a genetic risk factor of development of any cardiovascular disease.

### DESCRIPTION OF THE INVENTION

The aim of the present invention is detection of at least one allelic variant on the gene which codes for factor XII protein, this being very useful for genetic diagnosis since the heterozygote or homozygote individuals for the mutated allele are those who have a greater predisposition to suffer cardiovascular events. This represents a considerable advance, especially in the prevention of said cardiovascular diseases.

The present invention relates to a process for detecting the presence of at least one allelic variant in humans, said process includes the identification in the genetic material of an isolated biological sample from said human an allelic variant which consist on the substitution of a cytosine by an tymine in the position 46 over the transcriptional origin of Factor XII (46C/T) within the locus of chromosome 5, limited by the D5S400 and D5S408 markers, being the presence of said allelic variant is indicative of a predisposition to a cardiovascular disease.

The present invention is therefore directed at the identification of individuals who, not having yet developed the disease, constitute a risk group due to presenting at least one allelic variant on said chromosome locus, which makes them susceptible to developing a cardiovascular disease.

In one embodiment of the invention, the biological sample obtained from the human is, preferably, blood.

In the present invention, the term “genetic material” refers to the DNA sequence which is extracted from a biological sample. The extraction of DNA on the basis from the physiological sample can be carried out using any of the protocols known in the art (for example, the one described in the document Miller S A, Dykes D D, Polesky H F (1988) A simple salting out procedure for extracting DNA from human nucleated cells. Nucleic Acid Res 16: 1215).

In a preferred embodiment, the process of the present invention permits the identification of at least one allelic variant in the gene that codes for factor XII protein.

In the present invention, the term “allelic variant” refers to a genetic variation in the DNA sequence which codes for factor XII protein, said genetic variation involving a pathology, loss or gain of function. In particular, said genetic variation affects on susceptibility to suffering from a cardiovascular pathology.

The gene sequence that codes for protein factor XII in humans is described in many data banks, such as the OMIM data bank, in which the gene sequence which codes for protein factor XII has the access number 234000.004.

The genetic markers D5S400 and D5S408 are also described in several data banks, such as the Genome DataBank or the Data Bank of Human Genome. Said markers are focused on the positions 168.576. 667 bp (D5S400) and 180.015. 997 (D5S408) of chromosome 5 (numbering of pairs of bases (bp) from the beginning of the chromosome 5).

In another preferred embodiment of the present invention, the identification of at least one allelic variant in the genetic material of the biological sample includes prior to the identification, a polymerase chain reaction (PCR) is carried out in order to obtain a specific genomic fragment.

Detection of the presence of at least one allelic variant in the amplified fragment is carried out by any of the protocols known in the art, as for example by digesting the DNA fragment obtained by PCR in any of the restriction enzymes which gives rise to a differential pattern of electrophoretic bands in normal individuals, heterozygote carriers and homozygote carriers.

The present invention also refers to the use of a biological sample susceptible of including the allelic variant (46C/T) within the locus of chromosome 5 limited by the D5S400 and D5S408 markers in order to determine predisposition to cardiovascular diseases which manifest with thrombotic events.

There exists a considerable diversity of cardiovascular diseases which manifest with thrombotic events, such as acute myocardial infarct, ischaemic cerebrovascular accident, deep vein thrombosis, pulmonary embolisms, etc.

One advantageous aspect of the present invention is that it does not require special or complex techniques; indeed, the techniques used are generally known by any skilled in the art. The key aspect of the present invention is the detection of at least one allelic variant on a locus of chromosome 5 limited by the D5S400 and D5S408 markers in order to determine if there exists a predisposition to a cardiovascular disease in individuals who have not yet developed such a disease.

Moreover, with the process of the present invention it can be possible to identify genetic factors which yield understanding of the molecular bases of cardiovascular diseases, in particular those with thrombotic events, this being a key aspect for the development of more effective prophylactic and therapeutic processes.

Furthermore, the identification of one or more allelic variants in the gene sequence which codes for factor XII protein involves health-care advantages, since if an individual is identified as having an allelic variant in the gene which codes for factor XII protein, and that individual has not yet developed the pathology, a preventive and therapeutic strategy can be designed.

The present invention therefore permits the identification of gene loci which affect susceptibility to thrombosis and their intermediate phenotypes. It has the further advantage that, by allowing diagnosis in the initial phase of the disease, the mortality and morbility associated with thrombosis can be reduced.

There currently exists considerable interest among researchers in the field of molecular genetics in generating a list of all the genetic factors which contribute to the development of cardiovascular events.

Ideally, this list will help to increase knowledge of the mechanisms of formation of thrombi in a different variety of environments and to design treatment and prevention strategies specific to the genetic profile of the individual (Holtzman N A, Marteau T M. Will genetics revolutionize medicine?. N Engl J Med 2000 Julio 13; 343 (2): 141-4). The present invention constitutes an important step forward in the diagnosis and prevention of cardiovascular diseases.

There follows, by way of non-restrictive illustration, a description of an example of embodiment.

### EXAMPLES

Next, an example in which it is determined an allelic variant in the gene which codes for protein factor XII is enclosed.

1. Identification of an Allelic Variant in the Gene which Codes for Protein Factor XII

Firstly, samples of blood are taken from control subjects (250 healthy individuals) and patients (250 individuals to be diagnosed). Once the blood samples have been taken the DNA is extracted by means of any of the known standard protocols (Miller S A, Dykes D D, Polesky H F (1988) A simple salting out procedure for extracting DNA from human nucleated cells. Nucleic Acid Res 16: 1215). By use of the PCR technique, using standard conditions, the specific genomic fragment to be analyzed is obtained.

Secondly, at least one allelic variant is identified in the fragment obtained. Any of the protocols known in the art can be used for this purpose, such as direct sequencing of the amplified fragment, digestion with a restriction enzyme (as described above) or by specific hybridization probes marked with fluorescence.

In particular, and by way of illustration, an allelic variant will be determined in the gene which codes for the factor XII factor, known as 46 C/T due to its position in relation to the start of the transcription.

The diagnosis is based on analysis of the DNA molecule by PCR amplification of a genome fragment of 369 pairs of bases which contain the nucleotide 46 C/T, and digestion with the restriction enzyme SfaNI, which recognises the mutated sequence. The amplified fragment of a mutated allele is digested by SfaNI in fragments of 247 and 122 pb (Kanaji T, Okamura T, Osaki K, Kuroiwa M, Shimoda K, Hamasaki N, Niho Y (1998) A common genetic polymorphism (46 C to T substitution) in the 5′-untranslated region of the coagulation factor XII gene is associated with low translation efficiency and decrease in plasma factor XII level. Blood 91: 2010-2014).

Amplification by PCR: Fragment of 369 pb

Specific oligonucleotides:
Oligonucleotide 1: SEQ. ID NO: 1
Oligonucleotide 2: SEQ. ID NO: 2

PCR MIX (Promega master mix ref. M7502)

| | |
|---|---|
| Master mix | 12.5 μl |
| oligonucleotide | 12.5 μl |
| oligonucleotide | 22.5 μl |
| DNA | 4 μl |
| Water | 3.5 μl |
| Final volume: | 25 μl |

PCR program: Applied Biosystem PCR 9700

| | | |
|---|---|---|
| 5′ | 95 C. | |
| 1′ | 95 C. | |
| 1′ | 55 C. | ×30 cycles |
| 1′ | 72 C. | |
| 10′ | 72 C. | |

Detection: Digestion with the Enzyme SfaNI

Firstly, the reagents necessary for giving rise to digestion with the restriction enzyme SfaNI are defrosted. The following is then added in a tube:

5 μl of PCR product (without oil)*

0.1 μl SfaNI**

5 μl NEBuffer

0.5 μl BSA

H₂0 c. s. p. 50 μl

*from patients, normal heterozygote and homozygote control.

**the enzyme has to be always in an ace bath.

The mixture is shaken, centrifuged at 14000 rpm for 5 seconds and left in an oven at 37° C. overnight.

Finally, the reaction is halted at a temperature of 4° C. and then centrifuged at 14000 rpm for 5 seconds.

Results

—Expression of the Results:

An amplified fragment of 369 pb has to be obtained.

Identification of the digestion bands with the SfaNI enzyme of the normal alleles and of those carrying the allelic variant 46 C/T (mutated allele) is carried out by comparison with the pattern of bands of the Phi marker:

| | | Measurement of the bands |
|---|---|---|
| normal allele | C | 369 (1 band) |
| mutated allele | T | 247/122 (2 bands) |

—Interpretation of the Results of the Allelic Variant 46C/T:

The individuals in whom only one band of 369 pairs of bases is observed are homozygous for the normal allele, that is, they are carriers of two C alleles (one on each of the two chromosomes 5 wherein the gene which codes for factor XII protein is located).

The individuals in whom one band of 369 pairs of bases and another two of 247/122 pairs of bases is observed, respectively, are heterozygote for the normal and mutated allele, that is, they are carriers of the C allele (normal) on one of the chromosomes 5 and carriers of the allele T (mutated) on the other chromosome 5.

The individuals in whom only two bands are observed, one of 247 and another of 122 pairs of bases (the band of 369 pairs of bases is absent) are homozygote for the mutated allele, that is, they are carriers of two T alleles (mutated), one on each of the chromosomes 5 on which the gene coding for factor XII protein is located.

1-4. (canceled)

5. A method for detecting the presence of at least one allelic variant in a human, comprising the step of:
identifying, in genetic material of an isolated biological sample from a human, the allelic variant consisting of a substitution of a cytosine by a tymine in position 46 over a transcriptional origin of Factor XII (46C/T) within a locus of chromosome 5, limited by markers D5S400 and D5S408,
wherein the presence of the allelic variant is indicative of a predisposition to a cardiovascular disease.

6. The method according to claim 5, wherein the biological sample is blood.

7. The method according to claim 5, further comprising the step of:
before the identifying step, carrying out a polymerase chain reaction (PCR) in order to obtain a specific genomic fragment.

8. A method for determining predisposition of a human to a cardiovascular disease, comprising the steps of:
identifying, in genetic material of an isolated biological sample from a human, the allelic variant consisting of a substitution of a cytosine by a tymine in position 46 over a transcriptional origin of Factor XII (46C/T) within a locus of chromosome 5, limited by markers D5S400 and D5S408; and
determining a predisposition to a cardiovascular disease based on the allelic variant.

9. The method according to claim 8, wherein the biological sample is blood.

10. The method according to claim 8, further comprising the step of:
before the identifying step, carrying out a polymerase chain reaction (PCR) in order to obtain a specific genomic fragment
